(19) United States
(12) Patent Application Publication
(10) Pub. No.: US 2012/0172780 A1
(43) Pub. Date: Jul. 5, 2012

(54) SCROTAL SUPPORT GARMENT

(76) Inventor: DONALD R. QUINN, Bristol, TN (US)

(21) Appl. No.: 13/418,195

(22) Filed: Mar. 12, 2012

Related U.S. Application Data
(63) Continuation-in-part of application No. 12/071,878, filed on Feb. 27, 2008.

(51) Int. Cl. A61F 13/00

(52) U.S. Cl. 602/72

(57) ABSTRACT

The scrotal support garment includes a fabric material that forms a cup to support a wearer's scrotal sack and contents thereof. The cup has a wide upper end that tapers to a narrow lower end. The cup has at least one opening defined therein. An elongated thong extends from the lower end of the cup. The thong may be sewn to or bifurcate from the left and right shoulder straps. Elongated frontal straps extend from opposite sides of the cup. The shoulder straps and the frontal straps are joined by shoulder adjusters that provide height adjustment of the cup for comfortable support of a swollen or otherwise diseased scrotal sack.

### CROSS-REFERENCE TO RELATED APPLICATION

This application is a continuation-in-part of my prior U.S. patent application Ser. No. 12/071,878, filed Feb. 27, 2008.

### BACKGROUND OF THE INVENTION

1. Field of the Invention

The present invention relates to medical appliances and support devices for the male scrotum for patients having ailments or medical conditions causing the scrotum to become swollen and painful. The support device is intended to relieve pain and discomfort.

2. Description of the Related Art

Diseases such as penile cancer, testicular cancer, or the like and medical conditions resulting from a vasectomy, penectomy, or similar surgery can cause the male scrotum to swell or enlarge to an abnormal size. Further, conditions such as orchitis, epidyditmis, hydrocele, liver failure, and others may cause the scrotum to swell or enlarge to an abnormal size and resultant heaviness. An enlarged or swollen scrotum is usually very painful and can render such simple activities as walking, sitting, or standing unbearable. Moreover, the edematous scrotum may distress surrounding tissues and structures, which may lead to further damage. Although there are many devices for supporting the scrotum, none are entirely satisfactory for relieving the pain and discomfort since none transfer the weight of the scrotum to the shoulders where an enormous bulk of musculature is located.

Thus, a scrotal support garment solving the aforementioned problems is desired and necessary.

### SUMMARY OF THE INVENTION

The scrotal support garment includes a fabric material that forms a cup to support a wearer's scrotal sack and contents thereof. The cup may imperforate or have at least one opening for catheter and/or drainage tube access and patient cleaning. The opening in the cup may take the form of a flap opening or may be one or more apertures. An elongated thong extends from the lower end of the cup and may be made from the same or different fabric than the cup. The thong is preferably made of a more elastic fabric. The cup has a wide upper end that tapers to a narrow lower end. The top and sides of the cup may also be hemmed with elastic under tension in order to form a full cup that envelopes the swollen scrotum. The elongated thong may be integral with or separately attached to the lower end of the cup. In one embodiment, the thong bifurcates to form left and right shoulder straps that are unitary members extending to opposite sides of the upper end of the cup. In another embodiment, the thong is attached (e.g. sewn) to separate left and right shoulder straps In a further embodiment, the thong bifurcates to form a first portion of left and right shoulder straps. Elongated second portions of the left and right shoulder straps extend from opposite sides of the cup. The first and second portions are joined by a strap adjuster or releasable fasteners, such as buckles, so that the length of the shoulder straps is adjustable. In all the embodiments, the straps may be formed of the same fabric as the thong or be made from a more elastic material. The garment may be made in numerous sizes because of the plethora of conditions and degrees of edema.

These and other features of the present invention will become readily apparent upon further review of the following specification and drawings.

### BRIEF DESCRIPTION OF THE DRAWINGS

FIG. 1 is an environmental, perspective view of a first embodiment of a scrotal support garment according to the present invention as viewed from the front.

FIG. 2 is an environmental, perspective view of a scrotal support garment according to the present invention as viewed from the rear.

FIG. 3 is a perspective view of a second embodiment of a scrotal support garment according to the present invention.

FIG. 4 is a rear perspective view of a third embodiment of a scrotal support garment according to the present invention having an opening in the cup and shoulder pads attached to the shoulder straps.

FIG. 5 is a partial front perspective of the scrotal support garment of FIGS. 3 and 4, showing details of the cup portion of the garment.

FIG. 6 is a partial perspective view of a fourth embodiment of a scrotal support garment according to the present invention as seen from the rear, showing an opening in the cup having a flap.

Similar reference characters denote corresponding features consistently throughout the attached drawings.

### DETAILED DESCRIPTION OF THE PREFERRED EMBODIMENTS

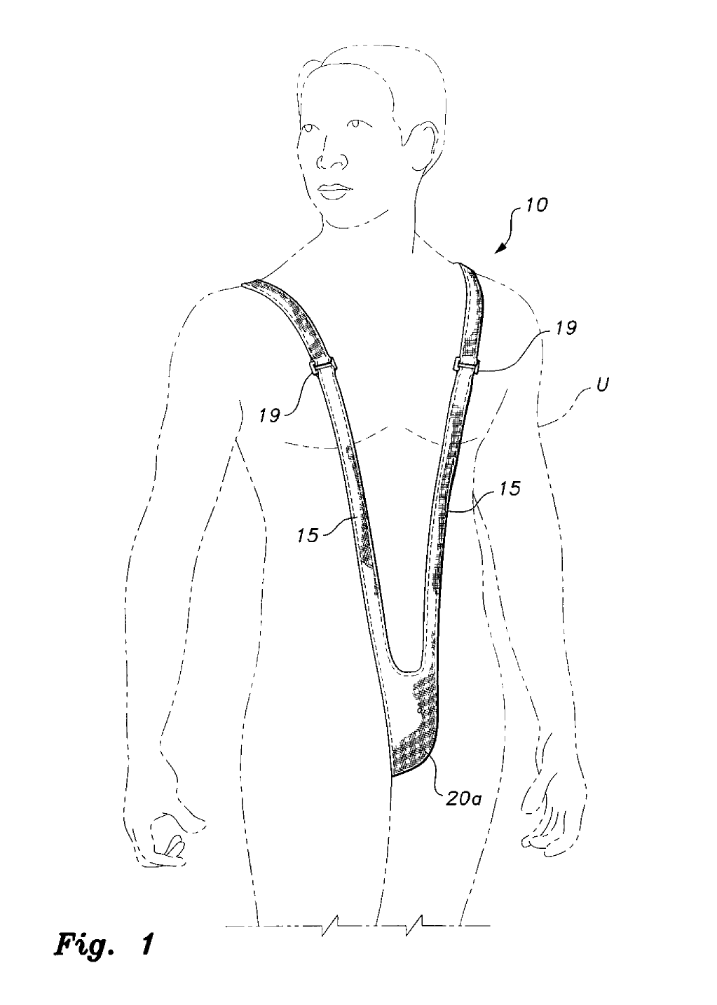

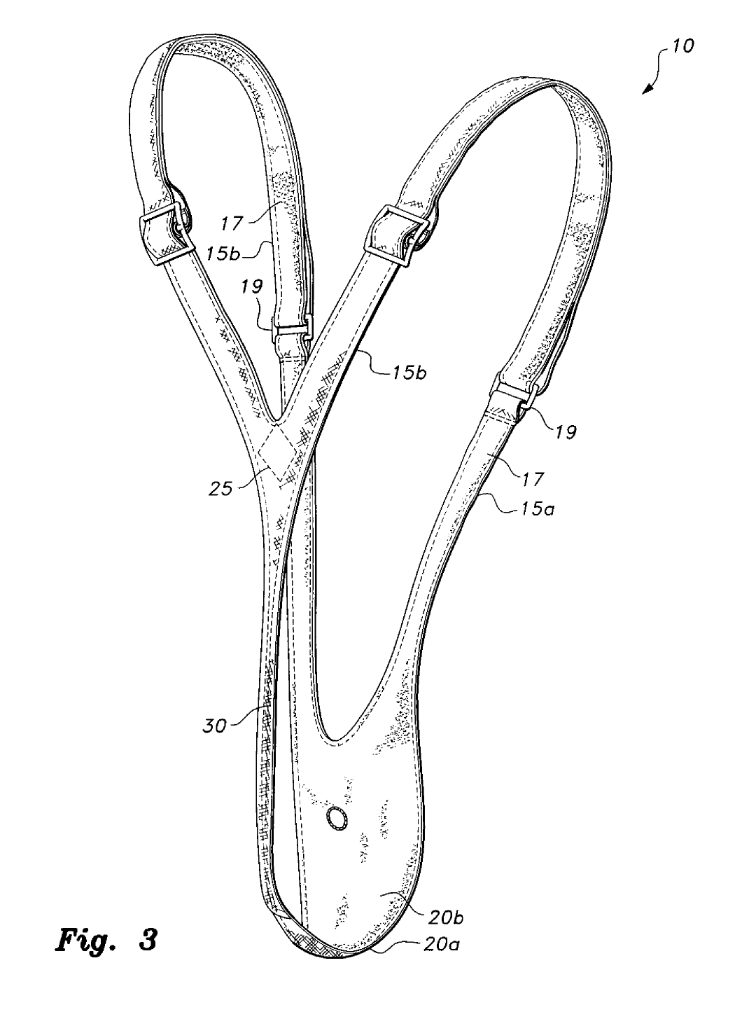

The scrotal support garment, as shown in FIG. 1, relates to a scrotal support garment 10 made from a flexible, preferably resilient material that forms a cup 20a. As shown in FIG. 3, an inside portion 20b of the cup 20a is adapted for holding an edematous, diseased, or otherwise swollen scrotal sack of the wearer U of the garment 10, thereby providing support of the scrotum. The cup inside portion 20b may have a liner made of an air permeable, liquid absorbent material to promote comfort and hygiene of the wearer. The cup material may be natural cloth, e.g., cotton, or it may be synthetic material, or a blend of cloth and synthetic material. The cup 20a has a wide upper end, the cup tapering to form a narrow lower end.

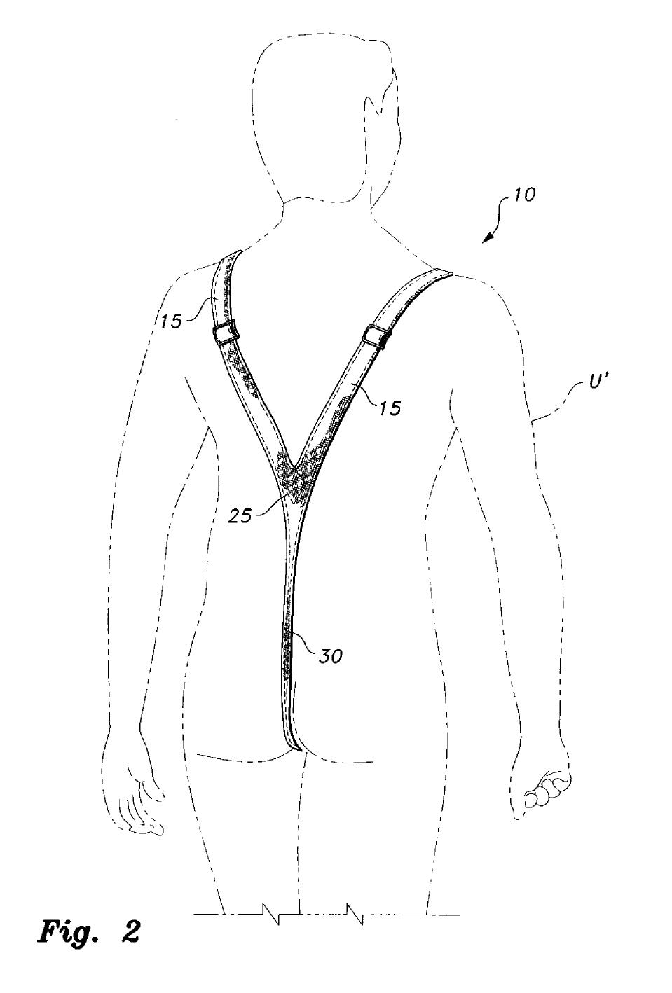

As shown in FIG. 2, an elongate, single thong 30 extends from the lower end of the cup 20a. The thong 30 bifurcates to form relatively slender left and right straps 15 that are attached to opposite sides of the upper end of the cup 20a. A diamond-shaped reinforcement 25 is stitched to the junction at which the shoulder straps bifurcate from the thong 30. The straps 15 may include means for adjusting the length of the straps, which may be shoulder adjuster loops and rings 19 joining front and back portions of the straps, buckles, mating fasteners, or any other means for adjusting the length of shoulder straps known in the art. The shoulder straps 15 may be lined with elastic strips 17 or bands (shown in FIG. 3). As shown in FIG. 3, the cup 20a preferably has at least one opening defined therein providing access for extending a catheter through the cup 20a, as described in more detail below. The straps 15 apply sufficient tension to the cup 20a to comfortably support an edematous, diseased, or otherwise swollen scrotal sack of the wearer U of the garment 10, thereby providing support of the scrotum.

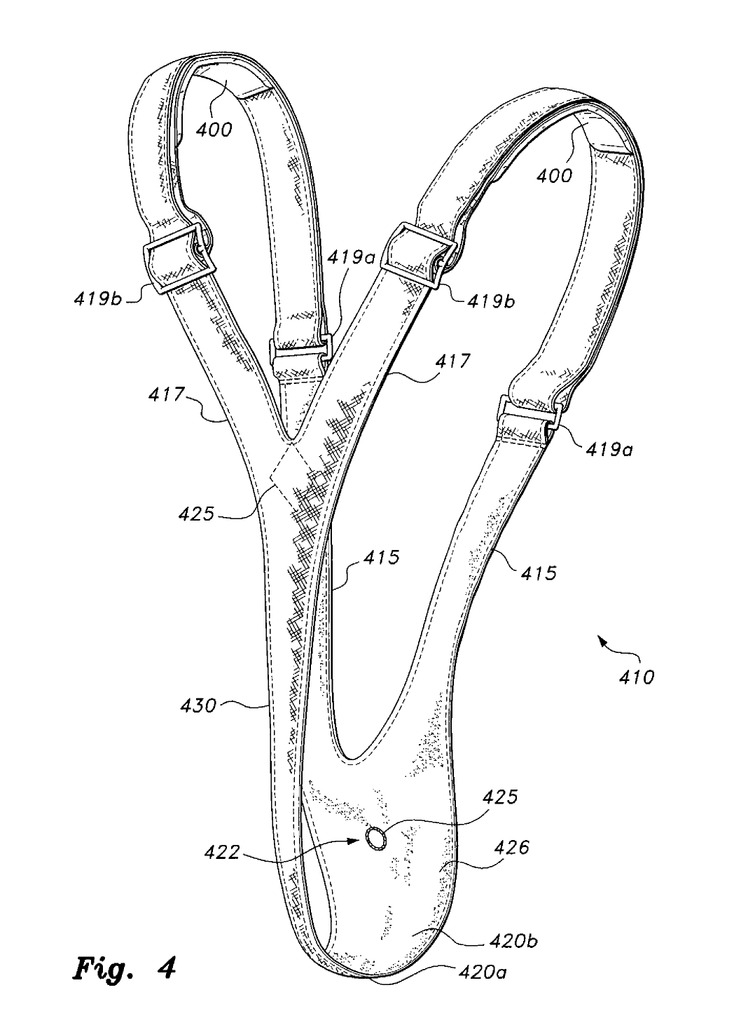

Referring now to FIG. 4, in an alternative embodiment, the scrotal support garment 410 includes a flexible, preferably resilient material forming a cup 420a, having an inside portion 420b that holds the scrotal sack of the wearer to support the scrotum. The cup inside portion 420b may include a liner material 426 made of an air permeable, liquid absorbent material to promote comfort and hygiene of the wearer. The cup material may be natural cloth, e.g., cotton, or it may be synthetic material, or a blend of cloth and synthetic material. The cup 420a has a wide upper end, the cup 420a tapering to form a narrow lower end. A wide, elongate, single thong 430 extends from the lower end of the cup 420a.

The thong 430 is elongated to form a single strap to extend up the user's back and bifurcates proximate and preferably above the small of the user's back to form relatively wide left and right over-the-shoulder straps 417 and front straps 415 joined by a shoulder adjuster mechanism for adjusting the length of the straps. In particular, each over-the-shoulder strap 417 is threaded under a lower post, over a center post, and under an upper post of dual ring adjustment retainer 419b. A bottom portion of the upper and lower posts of the dual ring adjustment retainer 419b may have teeth-like protrusions that frictionally assist in retaining an adjustment position of the over-the shoulder straps 417. Each over-the-shoulder strap 417 extends to loop around the upper post of a single ring adjustment retainer 419a, doubling back and being looped around the center post of the dual ring adjustment retainer 419b, where the loop is sewn in place to securely attach the end of the strap 417 to the center post. Relatively wide left and right frontal straps 415 extend from opposite sides of the cup's upper end, each frontal strap 415 looping around the lower post of the respective single ring adjustment retainer 419a, where the respective loop is sewn in place to securely attach the strap 415 to the lower post of the respective single ring adjustment retainer 419a. This arrangement permits adjustment of the height of the cup 420a by paying out or taking in the over-the-shoulder straps 417 at the dual ring adjustment retainer 419b. The straps 415, 417 may be made from elastic material, while the thong 430 may be made from a less elastic material. An optional diamond-shaped reinforcement 425 is stitched to the junction at which the shoulder straps 417 bifurcate from the thong 430. Instead of the diamond-shaped reinforcement, the elastic straps may cross and overlap each other, and be sewn together and then attached to the thong 430. The shoulder straps 417 may be lined with a padding material or gel 400 to facilitate shoulder comfort of the wearer. It is further seen that the thong 430 may or may not be the same material as the elastic straps 415,417 or cup 420a.

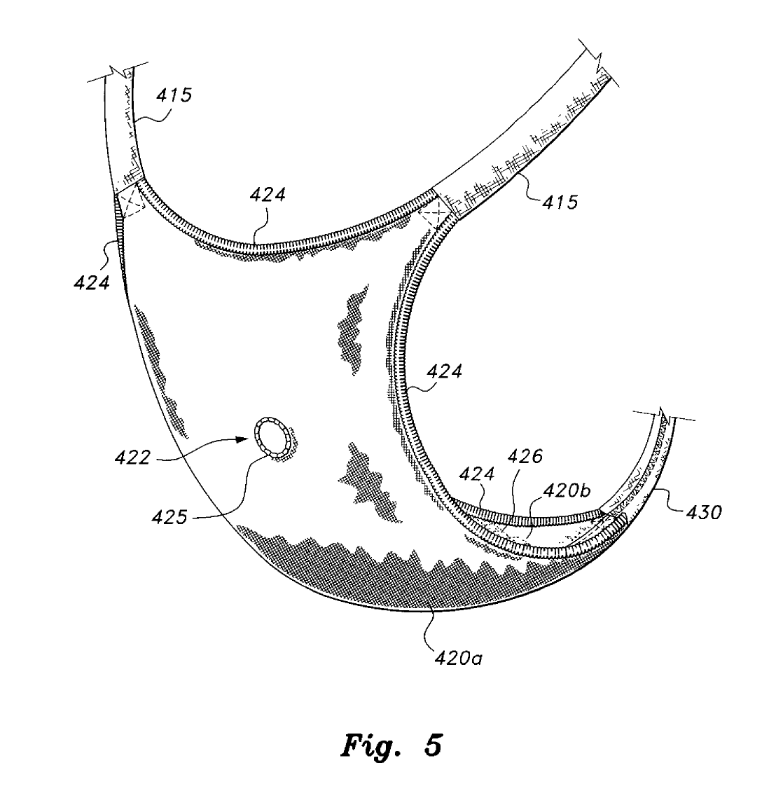

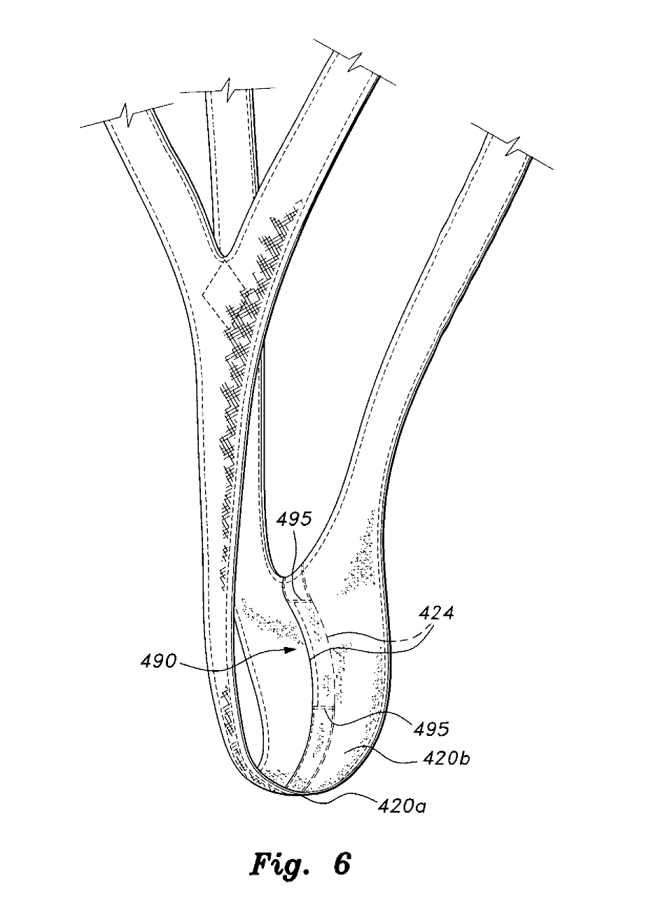

As most clearly shown in FIGS. 4 and 5, the scrotal supporting cup 420a of scrotal support garment 410 includes a circular catheter/drainage tube insertion opening 422 disposed on a front central portion of cup 420a. The opening may be, e.g., about one and one-half inches in circumference. A circular reinforcement hem 425 outlining the circumference of the catheter/drainage tube insertion opening 422 is provided to ensure against tearing or deformation of the insertion opening 422. Although a single opening is illustrated, one of ordinary skill in the art realizes that the number and location of the openings is variable (for example, an opening may be located at each of the upper portions of the cup at the widest part adjacent the straps 415) and dependent on the needs of the patient. Alternatively, as shown in FIG. 6, a flap 490 may be provided with elongate flap edges 424 and reinforcement stitching 495. The flap 490 can be opened for catheter/drainage tube cleaning and dressing wounds. The flap 490 may overlap the opposite side of the opening by about one inch, thereby covering the opening in the absence of any tension pulling the flap 490 away from the opposing edge of the opening. The flap 490 may optionally be provided with a reclosable fastener, e.g., a zipper or hook and loop fastener.

The peripheral edges of the cup 420a preferably have an elastic band 424 hemmed about the periphery of the cup 420a. Preferably, the elastic is placed under tension when it is hemmed into the garment thereby giving the cup more capacity for the scrotum. The hemmed elastic band 424 provides sufficient elasticity to the cup without the cup per se being highly elastic.

The garment straps 415 and 417 are wide (featuring an elongated width) and strong to better support the heavy edematous scrotum, and to minimize the pressure on the shoulder from the weight of the scrotum. The shoulder cushions or gel pads 400 may also be removable or provided in a kit as optional attachments to further minimize the stress on the shoulders for garments larger than 2-extra large, or if requested.

Because some patients are allergic to latex, the fabric is preferably latex-free. For hygienic reasons, and for those who perspire excessively or have leaking/seeping wounds or lymphedema, and to minimize the irritation caused by urinary leakage, the fabric is preferably breathable and quick drying. Therefore, the garment 410 is preferably made with a thick Lycra® (Lycra is a trademark of E.I. du Pont de Nemours and Company referring to spandex) that is stronger and thicker than regular spandex and offers less stretch than regular spandex to provide more support. The preferable Spandex®/polyester blend is approximately 87% polyester or Nylon®, the balance being Spandex®. Preferably, the density count of the fabric is 36 and the yarn count is 40D. The fabric is weft knitted.

It is to be understood that the present invention is not limited to the embodiments described above, but encompasses any and all embodiments within the scope of the following claims.

I claim:

1. A scrotal support garment, comprising:
a flexible, resilient cup adapted for supporting a scrotum, the cup having a wide upper end, the cup tapering to form a narrow lower end, the cup having at least one opening defined therein, the opening being dimensioned and configured for passage of a catheter/drainage tube through the opening;
an elongated thong extending from the lower end of the cup; and
left and right shoulder straps extending from the thong, the straps applying sufficient tension to the cup to comfortably support an edematous, diseased, or otherwise swollen scrotal sack of the wearer of the garment, thereby providing support of the scrotum.

2. The scrotal support garment according to claim 1, further comprising means for adjusting the length of the shoulder straps.

3. The scrotal support garment according to claim 1, wherein the at least one opening comprises a circular opening.

4. The scrotal support garment according to claim 3, wherein the thong and each of the straps are sewn together.

5. The scrotal support garment according to claim 3, further comprising a reinforcement hem forming a periphery around the circular opening.

6. The scrotal support garment according to claim 1, wherein further comprising a flap overlapping the at least one opening, the flap normally covering the opening.

7. The scrotal support garment according to claim 1, wherein said shoulder straps are made from an elastic fabric.

8. The scrotal support garment according to claim 1, wherein said cup is made from a fabric comprising approximately 87% polyester, the balance being spandex.

9. The scrotal support garment according to claim 8, wherein said shoulder straps are made from elastic material.

10. The scrotal support garment according to claim 1, further comprising shoulder pad members attached to said shoulder straps.

11. The scrotal support garment according to claim 10, wherein said shoulder pad members include cushion material.

12. The scrotal support garment according to claim 10, wherein said shoulder pad members are removably attached to said shoulder straps.

13. The scrotal support garment according to claim 1, wherein said cup defines a periphery, the scrotal support garment further comprising a tensioned elastic band hemmed around the periphery of said cup thereby increasing the capacity of the cup.

14. The scrotal support garment according to claim 1, wherein said thong has an extended portion adapted for extending. partially up the wearer's back, the extended portion terminating at a junction, the garment further comprising a reinforcement patch sewn to the junction, said shoulder straps being attached to the reinforcement patch.

15. A scrotal support garment, comprising:
a flexible, resilient cup adapted for supporting a scrotum, the cup having a wide upper end, the cup tapering to form a narrow lower end, the cup having at least one opening defined therein, the opening being dimensioned and configured for passage of a catheter/drainage tube through the opening;
an elongated thong extending from the lower end of the cup;
left and right shoulder straps extending from the thong, the straps applying sufficient tension to the cup to comfortably support an edematous, diseased, or otherwise swollen scrotal sack of the wearer of the garment, thereby providing support of the scrotum; and
means for adjusting the length of the shoulder straps.

16. The scrotal support garment according to claim 15, wherein said cup defines a periphery, the scrotal support garment further comprising an elastic band hemmed around the periphery of said cup.

17. The scrotal support garment according to claim 15, wherein said shoulder straps are made from elastic material.

18. A scrotal support garment, comprising:
a flexible, resilient cup adapted for supporting a scrotum, the cup having a wide upper end, the cup tapering to form a narrow lower end;
an elastic band hemmed around the periphery of the cup;
an elongated thong extending from the lower end of the cup;
left and right elastic shoulder straps extending from the thong, the straps applying sufficient tension to the cup to comfortably support an edematous, diseased, or otherwise swollen scrotal sack of the wearer of the garment, thereby providing support of the scrotum; and
a liner disposed in the cup, the liner being made from an air permeable, liquid absorbent material.

19. The scrotal support garment according to claim 18, wherein the junction of the thong and shoulder straps includes reinforcement material.

20. The scrotal support garment according to claim 18, further comprising means for adjusting the length of the shoulder straps.
